(19) United States
(12) Patent Application Publication
(10) Pub. No.: US 2007/0055007 A1
(43) Pub. Date: Mar. 8, 2007

(54) Weatherable corrosion-resistant flashing

(76) Inventors: Stephanie Pagel, Orono, MN (US); Paul Ludwig, Aurora, OH (US)

Correspondence Address: FISH & RICHARDSON P.C., PO BOX 1022, MINNEAPOLIS, MN, 55440-1022, US

(21) Appl. No.: 11/254,552

(22) Filed: Oct. 20, 2005

Related U.S. Application Data
(60) Provisional application No. 60/713,797, filed on Sep. 2, 2005.

(51) Int. Cl. A61L 2/08

(52) U.S. Cl. 524/569

(57) ABSTRACT

A corrosion resistant flashing product may comprise a polymeric composition that is substantially weatherable such that the flashing substantially retains its original properties after prolonged exposure to the outdoor elements while also providing substantial resistance to ACQ or CBA corrosives. In a preferred embodiment, the polymeric material comprises one or more light stabilizers from about 0.3% to about 15% by weight; one or more acrylic impact modifiers and/or process aids from about 2.5% to about 10% by weight; one or more thermal stabilizers from about 0.25% to about 3% by weight; one or more waxes or lubricants (or systems) from about 0.3% to about 7% by weight, and one or more plasticizers from about 0.1% to about 10% by weight.

### CROSS-REFERENCE TO RELATED APPLICATIONS

This application claims priority under 35 USC § 19(e) to U.S. Patent Application Ser. No. 60/713,797, filed by Stephanie Pagel on Sep. 2, 2005, the entire contents of which are hereby incorporated by reference.

### BACKGROUND

During construction of certain structures, precautions are taken to prevent water from seeping into areas in which mold or rot may occur. For example, flashing may be placed over a ledger board when constructing a deck for a house. If water were to penetrate behind the ledger board and into the side of the house, it may cause wood materials to rot, mold growth, and rust damage to the attachment hardware. Such circumstances can lead to structural failure of the deck and perhaps a portion of the house.

Many structures that are constructed for residential or commercial purposes may use treated lumber or other materials that can corrode to adjacent hardware. For example, in the past some chemically treated (or pressure-treated) lumber was treated with CCA (chromated copper arsenate). Certain metallic materials, such as aluminum or galvanized hardware, are resistant to the CCA corrosive. Thus, when constructing a structure with CCA-treated lumber, aluminum or lower-grade galvanization (zinc-coated) flashing products could be used.

The EPA (Environmental Protection Agency) has since banned the use CCA-treated lumber as a preservative for wood intended for residential use (except in limited circumstances). Other types of chemicals have been used to produce pressure-treated lumber within the EPA standard. For example, some modern pressure-treated lumber is treated with a chemical known as ACQ (alkaline copper quat) or CBA (copper azole). Like some other types of construction materials, the ACQ-treated or CBA-treated lumber contains a much higher concentration of copper, which can cause corrosion between dissimilar metals that are in contact with the lumber. Accordingly, much of the flashing hardware used in conjunction with the modern pressure-treated lumber or other construction materials comprises a heavily galvanized steel or copper sheet metal.

### SUMMARY

A corrosion resistant flashing product may comprise a polymeric composition that is substantially weatherable such that the flashing substantially retains its original properties after prolonged exposure to the outdoor elements while also providing substantial resistance to ACQ or CBA corrosives. In various embodiments, the composition may include a polymer selected from the group consisting of Acrylonitrile Butadiene Styrene (ABS), Polyvinylidinefluoride (PVDF), Nylon, Polycarbonate, Polycarbonate/ABS alloys, Polypropylene, and Polyvinylchloride (PVC). PVC is a preferred polymer. In particular embodiments, the flashing further comprises the following additives: light stabilizers, impact modifiers and processing aids, thermal stabilizers, fillers, waxes and lubricants, plasticizers, and pigments or colorants. In a preferred embodiment, the nonmetallic flashing sheet comprising a polymeric material, wherein the polymeric material comprises PVC from about 60% to about 90% by weight, a light stabilizer from about 3% to about 12% by weight, an impact modifier and/or processing aid from about 2.5% to about 10% by weight, a thermal stabilizer from about 0.25% to about 3% by weight wax or lubricant additive (or system) from about 0.3% to about 7% by weight, a plasticizer from about 5% to about 10% by weight, wherein the polymeric material has a tensile modulus of from about 266,000 to about 494,000 psi. In an alternate embodiment, the polymeric material comprises one or more light stabilizers from about 0.3% to about 15% by weight; one or more acrylic impact modifiers and/or process aids from about 2.5% to about 10% by weight; one or more thermal stabilizers from about 0.25% to about 3% by weight; one or more waxes or lubricants (or systems) from about 0.3% to about 7% by weight, and one or more plasticizers from about 0.1% to about 10% by weight.

Certain embodiments may provide one or more of the following advantages. First, the nonmetallic flashing equipment may be capable of contacting chemically treated lumber or other materials for a prolonged period of time without corrosion. Second, some embodiments of the nonmetallic flashing equipment may be capable of enduring the outdoor weather conditions for a prolonged period without substantially degrading. Third, some embodiments of the nonmetallic flashing equipment may be capable of retaining their original color or appearance for a prolonged exposure to sunlight and other outdoor elements. Fourth, some embodiments of the flashing equipment may have an appearance (e.g., a matte finish, a certain color, a wood grain finish, or the like) on one side that is different from the appearance (e.g., a glossy finish, a different color, or the like) of the other side. Fifth, some embodiments of the flashing equipment may comprise a material that can be cut to a desired shape using simple tools (e.g., a cutting blade or box cutter tool) at a jobsite. Sixth, some embodiments of the nonmetallic flashing equipment may comprise a material that is substantially flexible so that it may be readily supplied in a rolled condition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a portion of structure assembly in accordance with some embodiments of the invention.

FIG. 2 is a perspective view of a roll of nonmetallic flashing material in accordance with some embodiments of the invention.

FIG. 3 is a perspective view is a nonmetallic flashing device in accordance with some embodiments of the invention.

FIGS. 4-6 are perspective views of a portion of a process for constructing a structure assembly.

Like reference symbols in the various drawings indicate like elements.

### DETAILED DESCRIPTION OF ILLUSTRATIVE EMBODIMENTS

The construction of structures for residential or commercial purposes often requires the use of flashing equipment to prevent seepage of water into undesirable locations. Some embodiments include nonmetallic flashing equipment, for example, flashing equipment that comprises a polymeric material capable of operating in outdoor weather climates and capable of substantially retaining its original color after prolonged exposure to the outdoor elements. (As used herein, the term “nonmetallic” means not comprised primarily of metal) In certain embodiments, the nonmetallic flashing equipment may be used in conjunction with chemically treated lumber or other construction materials without inducing chemical reactions that lead to corrosion.

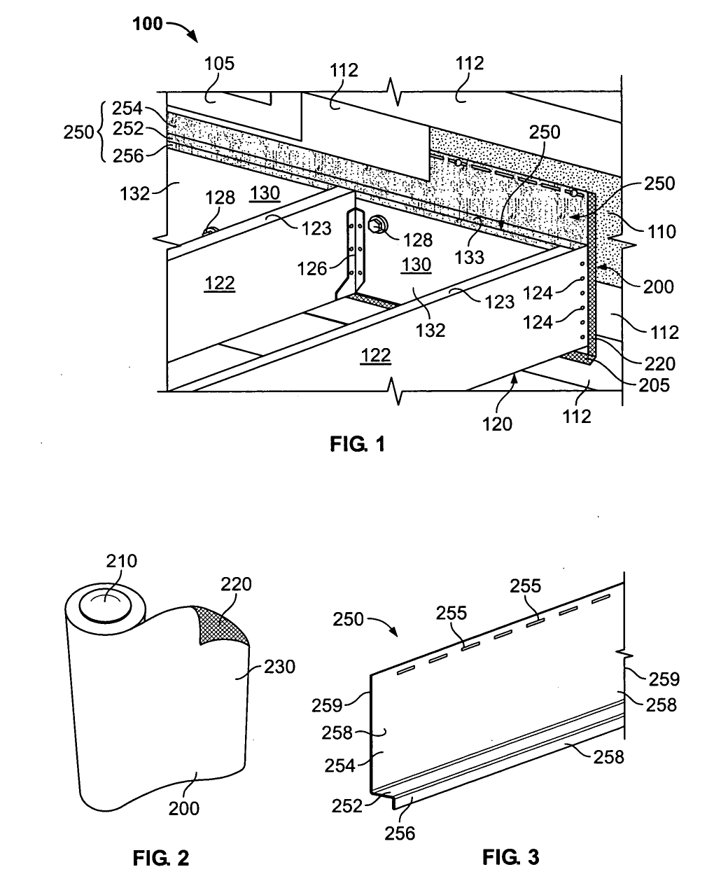

Referring to FIG. 1, a structure assembly 100 may employ flashing devices 200 and 250 to prevent water or other liquids from seeping into particular areas. In this embodiment, the structure assembly 100 includes a wall 110 of a house or other building and a deck structure 120 that mounted to the wall 110. It should be understood, however, that structure assemblies other than that shown in FIG. 1 may employ the nonmetallic flashing devices 200 and 250 described herein.

Flashing products having the configuration of flashing 200 may be referred to as “roll stock” flashing, in reference to the fact that the flashing may be advantageously provided in a form of a roll as described in further detail below. Flashing products having the configuration of flashing 250 are known in the industry as J channel flashing, in reference the to fact that the vertical section 254 and horizontal section 252 form generally a “J” shape.

The wall 110 may be a substantially vertical structure that extends upward from a ground surface. One or more pieces of siding members 112 may be attached to the wall 110 so that the siding members 112 facing outwardly from the wall 110. It should be understood that, in the illustration depicted in FIG. 1, a portion of one siding member 112 has been removed to better view the wall 110 and the nonmetallic flashing devices 200 and 250. The siding members 112 may overlap one another so that outdoor precipitation and other liquids do not pass through the siding members 112 and do not migrate into the wall 110.

At least a portion of the deck structure 120 may be mounted to the wall 110 so as to form the structure assembly 100. The deck structure 120 may include a plurality of joists 122 that extend from at least one ledger board 130 in a direction away from the wall 100. The joists 122 may be mounted to the ledger board 130 using fasteners such as nails 124, joist hanger devices 126, or a combination thereof. The joists may include a top surface 123 on which a plurality of a deck boards (not shown in FIG. 1) may be placed. As such, the deck structure 120 may be positioned relative to the wall 110 so that a person may step away from a door sill 105 and onto a plurality of a deck boards that collectively form a deck surface.

The ledger board 130, the joists 122, or a combination thereof may comprise wood material, such as cedar, pine, or a chemically treated lumber. For example, the ledger board 130 may comprise one or more, 2×12″ treated boards, 2×10″ treated boards, 2×8″ treated boards or the like. In another example, the joists may comprise 2×12″ treated boards, 2×10″ treated boards, 2×8″ treated boards or the like.

In some circumstances, the ledger board 130, the joists 122, or a combination thereof may comprise lumber that is chemically treated with a chemical known as ACQ (alkaline copper quat) or CBA (copper azole). The ACQ-treated or CBA-treated lumber may contains a concentration of copper that is sufficient to corrode some metallic hardware in contact with the lumber. As described in more detail below, the structure assembly 100 described herein may employ nonmetallic flashing devices 200 and 250 that are substantially resistant to corrosion from the chemically treated lumber.

Still referring to FIG. 1, the ledger board 130 may be mounted to the wall 110 so that an outside surface 132 of the ledger board 130 faces away from the wall 110. The ledger board 130 may be mounted to the wall 110 using one or more fasteners, such as lag bolts 128, nails, or the like. In some embodiments, at least a portion of the deck structure 120 may be supported by the attachment of the ledger board 130 to the wall 110.

If water or another liquid was permitted to penetrate between the ledger board 130 and the wall 110, the conditions would promote wood rot (e.g., rot of the ledger board 130 or wood materials in the wall 110), mold growth, and rust damage to the attachment hardware (e.g., rust damage to the fasteners such as lag bolts 128). Such circumstances can lead to structural failure of the deck and perhaps a portion of the house. To prevent the water or other liquid from penetrating between the ledger board 130 and the wall, the structure assembly 100 may include one or more nonmetallic flashing devices. In the embodiment shown in FIG. 1, the structure assembly 100 includes a nonmetallic flashing sheet 200 disposed between the ledger board 130 and the wall 10 and a nonmetallic flashing device 250 disposed over the top surface 133 of the ledger board 130 (e.g., to cover the area where the top surface 133 of the ledger board 130 meets the wall 110). In other embodiments, the structure assembly 100 may employ one of the flashing devices 200 or 250 to prevent the penetration of water between the ledger board 130 and the wall 110.

In the embodiment shown in FIG. 1, the ledger board 130 includes a single-width board. In such circumstances, the nonmetallic flashing device 250 may be configured to fit over the top side of the single-width ledger board. In other embodiments, the ledger board 130 may comprise two or more boards that are secured together along their abutting major surfaces. In those circumstances, the flashing device may be configured to fit over the double-width ledger board (or triple-width or the like).

Still referring to FIG. 1, the nonmetallic flashing sheet 200 may comprises a substantially flat sheet of material that can be cut to a predetermined size and disposed between the ledger board 130 and the wall 110. The nonmetallic flashing sheet 200 may be foldable or bendable so as to create a fold line or a bend in the sheet. As such, the nonmetallic flashing sheet 200 may be shaped to a desired configuration at the jobsite. For example, the nonmetallic flashing sheet 200 may be rolled or bent to include a lip portion 205 that extends away from the wall 110. Such a configuration may create a drip channel in which water or another liquid may drip from nonmetallic flashing sheet 200 to the ground without necessarily flowing along the nearby siding member 112 along the wall 110. Moreover, as described in more detail below, the nonmetallic flashing sheet 200 may comprise a polymer material that can be cut to a desired length or shape at the jobsite. For example, a user may employ a simple cutting blade (e.g., a box cutter tool) to trim the nonmetallic flashing sheet 200 to the proper length and shape.

Referring now to FIG. 2, the nonmetallic flashing sheet 200 may comprise a material that is substantially flexible so that it may be supplied in a rolled condition. When the nonmetallic flashing sheet 200 prepared for use at a jobsite (e.g., during the construction of the structure assembly 100), the nonmetallic flashing sheet 200 may be unrolled from a spindle 210 and cut to a desired length. For example, if the design of the structure assembly calls for a flashing sheet that is three feet, five feet, ten feet, twenty feet, forty feet, or more in length, the user may be able to bring only a single roll of nonmetallic flashing sheet to the jobsite and to cut the nonmetallic flashing sheet 200 to the desired length.

Referring to FIGS. 2 and 3, the flashing sheet 200 and J channel flashing 250 may comprise a nonmetallic material, such as a polymeric material, that is capable of enduring the outdoor weather conditions normally experienced by an exterior of a house or a building. For example, the nonmetallic flashing 200/250 may comprise a polymeric material having substantially low thermal expansion characteristics so that the nonmetallic flashing 200/250 does not excessively expand or contract due to varying temperature conditions. In certain embodiments, the nonmetallic flashing 200/250 may comprise a polymeric material that does not excessively contract beyond operability when exposed to temperatures of less than −30° F., less than −40° F., less than −60° F., less than −80° F., less than −100° F., less than −120° F., or less. In a number of embodiments, the nonmetallic flashing sheet 200/250 may comprise a polymeric material that does not excessively expand beyond operability when exposed to temperatures of more than 100° F., more than 120° F., more than 140° F., more than 160° F., more than 180° F., or more. Similarly, the nonmetallic flashing 200/250 may comprise a polymeric material that is capable of retaining its original color or appearance for a prolonged exposure to sunlight and other outdoor elements.

The polymeric material may include a polymer selected from the group consisting of Acrylonitrile Butadiene Styrene (ABS), Polyvinylidinefluoride (PVDF), Nylon, Polycarbonate, Polycarbonate/ABS alloys, Polypropylene, and Polyvinylchloride (PVC), and one or more additives. PVC is a preferred polymer. Typically, the polymer such as PVC will be from about 60% to about 90% by weight of the polymeric material. For example, the polymeric material can include about 70% to about 85% by weight, from about 75% to about 82% by weight, or from about 65% to about 75% by weight PVC. PVC and other polymers are commercially available, e.g., from Occidental, West Lake Chemicals, Georgia Gulf, etc.

The polymeric material can include one or more additives that can enhance or alter the light stability (including UV stability), weatherability, strength, thermal stability, rigidity, tensile and flexural properties, hardness, impact properties, aesthetic, and other qualities of the flashing material 200/250. Such properties can be evaluated using standard methods, e.g., ASTM methods (as described below). In addition, polymeric material stability can be evaluated using process, heat, and light stability tests, e.g., using a static oven test, a dynamic torque rheometer test, or a process-simulation test, such as milling. Weathering and light stability can be evaluated in a laboratory weathering chamber and exposed to light from xenon arc or fluorescent UV lamps. Moisture can be adjusted through humidity control.

A polymeric material can include, independently, one or more light stabilizers, impact modifiers and/or processing aids, thermal stabilizers, fillers, waxes and lubricants (e.g., wax or lubrication systems), pigments, or plasticizers, in various combinations and amounts. In certain embodiments, one, two, three, four, five, six, seven, eight, nine, ten, or more additives are added to the polymer to result in a desired polymeric material profile.

The particular selection of one or more additives can be chosen based on one or more characteristics of the profile desired for the polymeric material. For example, in certain embodiments, the polymeric material has a tensile modulus of from about 100,000 to about 500,000 psi, or any value therebetween. Thus, in some embodiments, the polymeric material has a tensile modulus of from about 200,000 psi to about 500,000 psi, from about 300,000 to about 450,000 psi, or from about 350,000 to about 400,000 psi, or, most preferably, from about 370,000 to about 390,000 psi. Tensile modulus can be evaluated using standard methods, e.g., ASTM D-638. In applications requiring stiffer materials, such as J channel products, the tensile modulus can be from about 300,000 psi to about 700,000 psi, or any value therebetween, e.g., from about 400,000 psi to about 600,000 psi, from about 500,000 psi to about 700,000 psi, from about 450,000 to about 550,000 psi, or, most preferably, from about 475,000 to about 525,000 psi.

The polymeric material can have a tensile strength at yield of from about 4000 to about 8500 psi, or any value therebetween. For example, in some embodiments, the polymeric material has a tensile strength at yield of from about 5000 to about 7300 psi, or from about 5500 to about 6800 psi, or from about 6000 to about 6500 psi. Tensile strength at yield can be evaluated using ASTM D-638.

The polymeric material can have a specific gravity, as measured by ASTM D-792, of about 1.1 to about 1.8, or any value therebetween, e.g., about 1.2 to about 1.6, about 1.3 to about 1.5, or about 1.4 to about 1.5. Hardness, Shore D, instantaneous, as measured with ASTM D-2240, can range from about 50 to about 100, or any value therebetween, e.g., about 60 to about 90, about 70 to about 80, about 72 to about 80, or about 75 to about 80.

Flexural properties include flexural strength and modulus, e.g., as measured with ASTM D-790. The polymeric material can thus have a flexural strength from about 8000 to about 14,000 psi, or any value therebetween, e.g., about 10,000 to about 13,000, or about 11,000 to about 12,000 psi. Flexural modulus can vary from about 270,000 to about 500,000 psi, or any value therebetween, e.g., from about 325,000 to about 450,000, or from about 350,000 to about 400,000 psi.

Impact properties can be assessed with ASTM D-256. For example, notched Izod can range from about 2.5 to about 6.0 ft.-lb./inch, or from about 3.0 to about 5.5 ft.-lb./inch, or from about 3.8 to about 4.4 ft.-lb./inch. Dart drop, Min. Failure in.-lb./mil, assessed using ASTM D-4226, can yield a value of from about 1.0 to about 2.0, or from about 1.3 to about 1.7, or from about 1.4 to about 1.6 using procedure A, and from about 4 to about 8, or from about 5 to about 7.5, or from about 5.5 to about 6.5, using procedure B.

With respect to thermal properties, the polymeric material can exhibit a heat deflection temperature at 264 psi, in ° F., as assessed with ASTM D-648, of about 115 to about 215, or from about 130 to about 200, or from about 145 to about 175, or from about 155 to about 168. The coefficient of linear expansion, as assessed with ASTM D-696, can range from about 2.5×10⁻⁵ in./in./° F. to about 4.8×10⁻⁵ in./in./° F., or from about 3.0×10⁻⁵ in./in./° F. to about 4.3×10⁻⁵ in./in./° F., or from about 3.3×10⁻⁵ in./in./° F. to about 4.2×10⁻⁵ in./in./° F.

Additives that can affect one or more of the properties described above can include light stabilizers. A light stabilizer can aid in weatherability, by e.g., absorbing light such as UV light. Examples of light stabilizers include hindered amine light stabilizers (HALS), 2-hydroxybenzophenones, and benzotriazoles. In certain embodiments, Titanium dioxide (TiO₂) can be used, which can also act as an opacifier or whitening agent.

One or more light stabilizers can be added in a total amount of from about 0.3% to about 15% by weight of the polymeric material, or any value therebetween. For example, HALS, substituted benzophenones, and benzotriazoles can be typically used from about 0.3% to about 4% by weight. TiO₂ can be included from about 0.3% to about 15% by weight, or from about 3% to about 12% by weight, or from about 5% to about 10% by weight, or from about 6% to about 8% by weight. HALS are available commercially, e.g., from Ciba-Geigy, Kerr-McGee, DuPont, Cytec, Chemtura and others, e.g., the Chimassorb® 944 family from Ciba-Geigy, the Tinuvin® 123family from Ciba-Geigy, and the Markscreen® from Chemtura. Substituted benzophenones are also commercially available, e.g., Ciba-Geigy's Chimassorb® 81 and Chemtura's Lowilite® families, as are Benzotriazoles, e.g., Ciba-Geigy's Tinuvin® 213, 234, 327, 328, and 571 families. Tinuvin® XT833 can also be used in certain applications. TiO2 is available commercially from Kerr-McGee, e.g., Tronox® R-FK-1 and R-FK-2, and other sources.

One or more impact modifiers and/or processing aids (e.g., acrylic processing aids) can also be included in the polymeric material to enhance impact strength, improve processing, and reduce brittleness. An impact modifier and/or processing aid can be from about 0% to about 10% by weight of the polymeric material or any range therebetween, e.g., about 1% to about 10%, about 2.5% to about 10%, about 3% to about 8%, or about 5% to about 7%.

An impact modifier can be an acrylic impact modifier or a chlorinated polyethylene impact modifier. Acrylic impact modifiers (AIM) can be core-shell impact modifiers, containing typically a shell of polymethylmethacrylate (PMMA) and a rubbery core of monomers of butyl acrylate or polymers of the same, e.g., polybutylacrylate (PBA). The PMMA shell can enable flowability and compatibility with PVC, while the butyl acrylate core provides impact strengthening. Acrylic impact modifiers and processing aids are commercially available, e.g., from Arkema (e.g., the Durastrength® 529, 510, 506, 400, 320, and 200 families of AIMs, and the Plastistrength® 530, 550, 551, 710, 770, and L1000 families of processing aids), Rohm & Haas (e.g., the Paraloid® K 100 and KM-369 families), LG Chemical (e.g., the IM808 and IM 808A families) and ChemicalLand21.com (e.g., AIM-07 and -08). Chlorinated polyethylene impact modifiers are also available commercially, e.g., from Dupont-Dow (the Tyrin 7000 and 2500 families).

A polymeric material can include one or more thermal stabilizers to aid in thermal stability during processing. Thermal stabilizers can prevent dehydrochlorination of PVC, prevent discoloration (e.g., yellowing, blackening), and improve stabilization of the resultant product. A thermal stabilizer can be included at from about 0.25% to about 3% by weight, e.g., from about 0.5% to about 2.5%, from about 1% to about 2%, or from about 0.75% to about 1.5%. Examples of thermal stabilizers include organotin compounds, such as octyl, methyl, and butyl tin and thioesters and mercaptides thereof, and octyl, methyl, and butyl tin carboxylates and tin maleates; mixed metal stabilizers, including zinc, calcium/zinc, magnesium/zinc, barium/zinc, barium/cadmium, and barium/calcium/zinc mixed metal stabilizers; and organic based stabilizers. A wide variety of organotin stabilizers are available commercially from Crompton (e.g., the Mark® 17 MLS, 17 MOK, 17 MOKD, 17 MOKN, 176 M, 1900, 1921, 1925, 1939, 1971, 1984E, 1987, 1998, 2208, 2270, 2284, 2289, 275, 2910, DBTL, T 150, T 201, T 216, T 22 M, T 22 M GV, T 267, T 634, T 682, and TK 262 GV families), Arkema (e.g., the Thermolite® T 108, T 490, T890F, T190, T31, T31W, T490, T890F, T892WF families), and Rohm and Haas (e.g., the Advastab® family). Mixed metal stabilizers are also available from Crompton (e.g., the Mark® 1034, 1221, 1495C, 3020, 3023, CZ 11, C 113, CZ 116, CZ 118 S, CZ 122, CZ 123, CZ 2000, CZ 400, CZ 97, EZ 760, and QTX families of Ca/Zn mixed metal stabilizers; the Mark® 4712, 4716, 4718, 4734, 4753, 4757, 4781A, 4823, 4830, 4835, 4843, 4844, 6705, 6711, 6717, 6726, 6729, 6731, 6734, 6736 ACM, 6749, 6750, 6751, 6767, and 9302 families of Ba/Zn mixed metal stabilizers; the Mark® 6092 ACM family of Mg/Zn mixed metal stabilizers; and the Mark® 3070 and Z2020 families of Zn metal stabilizers); Akzo Nobel (e.g., the Lankromark® LZB families of Ba/Zn mixed metal stabilizers and the Lankromark® LZC families of Ca/Zn mixed metal stabilizers); Ferro (e.g., the Therm-check® 840, 659, 7206, 7209, and 7710 families of Ca/Zn mixed metal stabilizers); and Rohm and Haas (e.g., the Advapak® family of Ca/Zn mixed metal stabilizers). Organic based stabilizers are available from Crompton (e.g., the Mark® A70, OBS 100 and OBS 200 families).

A polymeric material can include one or more fillers, e.g., to aid in processing and enhance weatherability. A filler can be one or more of calcium carbonate, such as precipitated calcium carbonate (available from Solvay), limestone, marble, talc, clay, wood chips, sea shells, diatomaceous earth, or other fillers known to those having ordinary skill in the art. One or more fillers can be added in an amount of from about 0% to about 50% by weight, e.g., from about 1% to about 25%, or from about 2% to about 15%, or from about 3% to about 10%, or from about 3% to about 8% by weight.

One or more wax or lubricant additives (or systems) can be included as an additive in a polymeric material described herein. One or more wax or lubricant additives or systems can be included in a total amount from about 0.3% to about 7% by weight, e.g., about 1% to about 5%, about 0.4% to about 3%, about 2% to about 5%, or about 0.5% to about 4%. A variety of waxes or lubricants, or combinations thereof, can be used. Examples include oxidized and unoxidized polyethylene (PE) based waxes, polypropylene based waxes, and paraffin-based waxes; metal soaps (e.g., calcium, zinc, barium, magnesium, lead, aluminum, sodium, tin, and cobalt stearate); primary and secondary amides (e.g., erucamide, oleamide, and stearamide, EBS and EBO); acid esters (e.g., PEMS, PEDS, PETS, PEAS, GMS, GMO, stearyl stearate, distearyl phthalate); and saturated and unsaturated fatty acids (lauric, myristic, palmitic, stearic, oleic, and erucic acids). A preferred wax or lubricant is a metal stearate, such as calcium stearate, in an amount of from about 0.4% to about 3.0%. Metal stearates can scavenge acid and enhance processability. Waxes and lubricants are available commercially from Ferro (e.g., the Petrac® 165, 215, and 480 Waxes and the Synpro® Calcium Stearate 12B, 15F, 91, and 92 families of calcium stearates), Baerlocher (the Ceasit® families of calcium stearates), Dover, and Crompton.

One or more plasticizers can also be included in a polymeric material. Plasticizer classes include phthalate derivatives, benzoate esters, and epoxidized vegetable oils. Useful plasticizers include DOP (dioctylphthalate), TBP (texanol benzyl phthalate), DINP (diisononyl phthalate), DIDP (diisodecyl phthalate), DOA (dioctyl adipate), TOTM (tris-2-ethylhexyl trimellitate), DEHP (diethylhexylphthalate), DBP (dibutylphthalate), DMP (dimethylphthalate), DEP (diethylphthalate), DUP (diundecylphthalate); epoxidized vegetable oils (soybean (ESO), linseed, crambe, and castor); and epoxidized methyl soyate (EMS) and allyl soyate (EAS). In certain embodiments, an epoxidized vegetable oil such as ESO is preferred. A plasticizer can be included in an amount from about 0.1% to about 10% by weight, or from about 0.1% to about 3%, 0.5% to about 5%, 1% to about 3%, 5% to about 10%, 7% to about 10%, 6% to about 8%, 8% to about 9%, 5% to about 9%, 2% to about 7%, 3% to about 6%, and 9% to about 10%. Plasticizers are available commercially from a variety of sources, e.g., Eastman Chemical, Ferro (e.g., the Santicizer® families of plasticizers), BASF (e.g., the Palatinol® and Plastomoll® families), Crompton, Chemtura, Dow (e.g., Flexol®) Epoxidized Soybean Oil) and Cognis.

Other additives for inclusion include pigments and colorants, flame retardants, antistatic agents, and antioxidants, as known to those having ordinary skill in the art.

In some embodiments, one or more of the described additives can be included in a polymeric material, e.g., a PVC polymeric material, to result in the flashing 200/250 having certain properties. For example, the flashing 200/250 can include a polymeric material having a plasticizer from about 5% to about 10% by weight or any value therebetween (e.g., 5%, 5.5%, 6%, 6.5%, 7%, 7.5%, 8%, 8.5%, 9%, 9.5%), and having a tensile modulus of from about 266,000 to about 494,000 psi, or any value therebetween, as discussed previously.

In some embodiments, the flashing 200/250 can include a polymeric material comprising one or more light stabilizers from about 0.3% to about 15% by weight; one or more acrylic impact modifiers and/or process aids from about 2.5% to about 10% by weight; one or more thermal stabilizers from about 0.25% to about 3% by weight; one or more waxes or lubricants from about 0.3% to about 7% by weight, and one or more plasticizers from about 0.1% to about 10% by weight.

Referring again to FIG. 2, the roll of nonmetallic flashing 200/250 may be manufactured using a flat-sheet extrusion process. In such circumstances, the heated polymeric material or other extrudable material is forced through one or more rollers, dies or a combination thereof as the material is cooled so as to produce a length of flat sheet material that can be rolled onto a spindle 210. In some embodiments, the roll of nonmetallic flashing sheet material may be manufactured in different heights so that a user can select a particular roll height that is sufficient for his or her purposes. For example, the roll of nonmetallic flashing sheet material may be manufactured to have heights of four inches, six inches, eight inches, ten inches, twelve inches, fourteen inches, sixteen inches, twenty inches, twenty-four inches, or greater. As such, a user may select the roll that has the appropriate height for the particular structure assembly 100 (FIG. 1) being constructed and then merely cut the desired length of the nonmetallic flashing sheet 200 at the jobsite.

The nonmetallic flashing 200/250 may be manufactured so that a first side has a different appearance than the opposing second side. For example, the nonmetallic flashing sheet 200 may comprise a material (and, in some embodiments, may comprises pigments or additives embedded in proximity to one side) or may be manufactured with specially configured rollers, dies, and other equipment so that the first side 220 has a substantially matte finish and the second side 230 has a substantially glossy finish. For example, rollers, such as chromium plated rollers, having various root mean squared (rms) values (microinch) can be employed, including 35 rms rollers, glossy rollers, etc., as known by one having ordinary skill in the art. As such, a user may decide whether a matte finish or a glossy finish provides the most desirable appearance. In some embodiments, a matte finish having an rms surface roughness of about 25 to about 50, or from about 30 to about 40, or about 35 microinch is preferred. Surface texture or roughness can be analyzed using standard techniques known to those having ordinary skill in the art, e.g., interferometer methods using diamond tip styluses, etc.

It should be noted that surface finish may affect the ability of the flashing to wick water away from a joint area. Glossier finishes tend evacuate water better than matte finishes. Accordingly, the provision of a matte finish is particularly desirable in applications where aesthetic concerns predominate over evacuation performance, such as residential applications.

In another example, the nonmetallic flashing 200/250 may comprise a material (and, in some embodiments, may comprises pigments or additives embedded in proximity to one side) or may be manufactured with specially configured equipment so that the first side 220 has color that is different from the color of the second side 230. In such circumstances, the user may decide which color is most appropriate when the material is at the actual jobsite.

In a further example, the nonmetallic flashing 200/250 may comprise a material (and, in some embodiments, may comprises pigments or additives embedded in proximity to one side) or may be manufactured with specially configured rollers, dies, and other equipment so that the first side has a wood grain finish and the second side has another color (e.g., a neutral color). Again, in such circumstances, a user may decide whether the wood grain finish would benefit the appearance of the structure assembly 100 (FIG. 1) or whether the color on the second side 230 would provide a better appearance.

Referring now to FIG. 3, the nonmetallic flashing device 250 may comprise a length of material that is configured to rest over with a ledger board 130 in a predetermined orientation. The nonmetallic flashing device 250 may comprise a substantially horizontal section 252 that is configured to be disposed adjacent to the top surface 233 (FIG. 1) of the ledger board 130. Further, the nonmetallic flashing device 250 may comprise a first substantially vertical section 254 extends upwardly from the horizontal section 252. The first vertical section 254 is configured to abut the wall 110 (FIG. 1) or another surface to which the ledger board 130 is attached. In some embodiments, the first vertical section 254 may include slots 255 or holes through which a nail or other fastener may pass so as to mount the flashing device 250 to the wall 110 or other surface. Also, the nonmetallic flashing device 250 may comprise a second substantially vertical section 256 that extends downwardly from the horizontal section 252 (oppositely the first vertical section 254). The second vertical section 256 is configured to be disposed adjacent to the outside surface 132 (FIG. 1) of the ledger board 130. As such, when the horizontal section 252 rests on the top surface 133 of the ledger board (FIG. 1), the nonmetallic flashing device 250 substantially covers the interface between the ledger board 130 and the wall 110 so that water or other liquids are prevented from penetrating therebetween.

The flashing device 250 may comprise a nonmetallic material, such as a polymeric material described previously, that is capable of enduring the outdoor weather conditions normally experienced by an exterior of a house or a building. Similar to the flashing sheet 200 previously described, the nonmetallic flashing device 250 may comprise a polymeric material having desired characteristics with respect to, e.g., weatherability, strength, rigidity, durability, and other characteristics as described previously. For example, the nonmetallic flashing device 250 may comprise a polymeric material having one or more additives as described previously, in any combination or amount as described previously. The particular selection of one or more additives can be chosen based on one or more characteristics of the profile desired for the polymeric material. For example, in certain embodiments, the polymeric material has a tensile modulus of from about 350,000 psi to about 600,000 psi, or any value therebetween, e.g., from about 400,000 to about 500,000 psi, from about 420,000 to about 550,000 psi, from about 475,000 to about 575,000 psi, or from about 350,000 psi to about 500,000 psi.

Still referring to FIG. 3, the length of nonmetallic flashing device may be manufactured using a profile extrusion process. In such circumstances, the heated polymer or other extrudable material is forced through one or more dies as the material is cooled so as to produce a length of material have the profile shape as previously described. In some embodiments, nonmetallic flashing device 250 may be manufactured in different lengths so that a user can select a particular length that is sufficient for his or her purposes. For example, the nonmetallic flashing device 250 may be manufactured to have lengths of four feet, six feet, eight feet, twelve feet, sixteen feet, twenty feet, twenty-four feet, thirty-six feet, forty-eight feet, or greater. As such, a user may select the length or lengths that are appropriate for the particular structure assembly 100 (FIG. 1) being constructed. The nonmetallic flashing device may be cut to the exact length at the jobsite if it is different from one of the manufactured lengths. For example, a user may employ a handheld cutting blade (e.g., a box cutter tool) to trim the nonmetallic flashing device 250 to the proper length and shape.

The nonmetallic flashing device 250 may be manufactured so that a first side 258 has a different appearance than the opposing second side 259. For example, the nonmetallic flashing device 250 may comprise a material (and, in some embodiments, may comprises pigments or additives embedded in proximity to one side) or may be manufactured with specially configured rollers, dies, and other equipment so that the first side 258 has a substantially matte finish and the second side 259 has a substantially glossy finish. In another example, the nonmetallic flashing device 250 may comprise a material (and, in some embodiments, may comprises pigments or additives embedded in proximity to one side) or may be manufactured with specially configured equipment so that the first side 258 has color that is different from the color of the second side 259. In a further example, the nonmetallic flashing device 250 may comprise a material (and, in some embodiments, may comprises pigments or additives embedded in proximity to one side) or may be manufactured with specially configured rollers, dies, and other equipment so that the first side 258 has a wood grain finish and the second side 259 has another color (e.g., a neutral color). In these circumstances, the costs of manufacturing may be reduced by producing the selected appearance effects only on the first side 258 of the device 250.

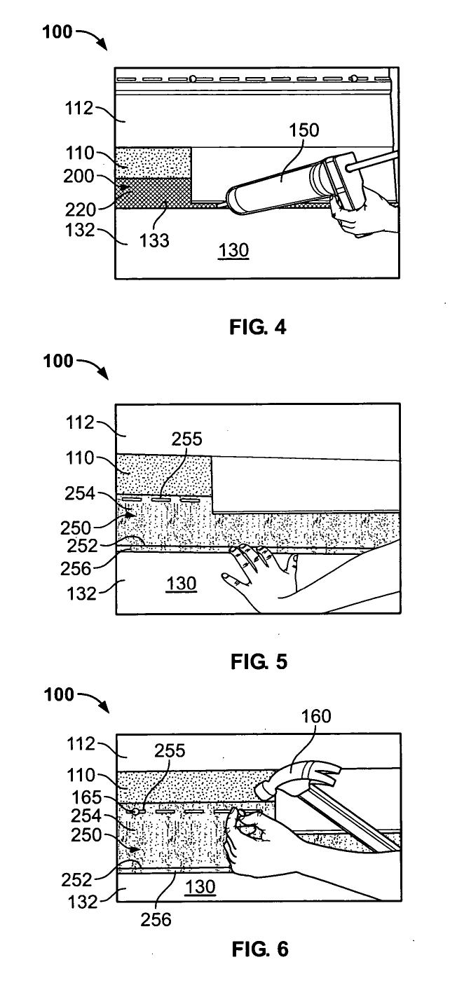

Referring now to FIGS. 4-6, a process for constructing a structure assembly 100 (FIG. 1) may include mounting a ledger board 130 to a wall 110. As previously described, the wall 110 may have one or more siding members 112 attached thereto. It should be understood that a portion of one more siding members 112 may be removed to reveal the wall 110 in the area where the ledger board 130 will be mounted. At least a portion of the siding members 112 may be reattached after the flashing sheets 200 and flashing device 250 are installed.

Referring to FIG. 4, the nonmetallic flashing sheet 200 may be cut to proper length and secured to the wall 110 in the area in which the ledger board 130 will be mounted. The ledger board 130 may then be mounted to the wall 110 so that the nonmetallic flashing sheet 200 is disposed between the ledger board 130 and the wall 110. As previously described, the ledger board 130 may be mounted to the wall 110 using one or more fasteners such as lag bolts 128. In some embodiments, the process may include applying an adhesive or sealant, such as silicone or caulk 150, in the crevice between the ledger board 130 and the wall 110 (proximal to the top surface 133 of the ledger board 130).

Referring now to FIG. 5, the nonmetallic flashing device 250 may be installed so that the substantially horizontal section 252 abuts the top surface 133 (FIG. 4) of the ledger board 130. In such circumstances, the first substantially vertical section 254 may be disposed proximal to the wall 110 or another vertical surface, as previously described. If some portion of the siding member 112 overlaps with the first vertical section 254, the nonmetallic flashing device 250 may be installed so that the first vertical section 256 fits behind the siding member 112 (as shown on the right side of the illustration in FIG. 5). When the horizontal section 252 is disposed on the top surface 133 of the ledger board 130, the second substantially vertical section 256 may be disposed proximal to the outside surface 132 of the ledger board 130.

Referring to FIG. 6, the nonmetallic flashing device 250 may be secured to the wall 110 using, for example, fasteners 165 passing through slots 255 in the first vertical section 254. In this embodiment, a user may employ a hammer device 160 to force fasteners 165 (e.g. nails) through the slots 255 and into the wall 110. As such, the nonmetallic flashing device 250 may be secured to the wall 100 before additional siding members 112 are attached to conceal the upper portion of the nonmetallic flashing device 250. When the nonmetallic flashing sheets 200 and nonmetallic flashing device 250 are installed, this configuration prevents water or other liquid from penetrating into the crevice between the ledger board 130 and the wall 110 and directs the water or other liquid to the outside surface 132 of the ledger board 130.

In other embodiments, the nonmetallic flashing sheet 200 or the nonmetallic flashing device 250 may be installed individually. For example, some structure assemblies 100 (FIG. 1) may not require a nonmetallic flashing sheet 200 behind the ledger board 130. Rather, the ledger board 130 may directly abut the wall 110 and the nonmetallic flashing device 250 may be installed over the top surface 133 of the ledger board 130, for example, similar to the process shown in FIGS. 5-6). Alternatively, the structure assembly may require only the nonmetallic flashing sheet 200 so that a top portion is fastened to the wall 110. In those circumstances, the lower portion of the sheet 200 may hang over the top surface 133 of the ledger board and abut against the outside surface 132 of the ledger board 130.

In some other embodiments, structure assemblies other than that shown in FIG. 1 may employ the nonmetallic flashing sheets and devices 200 and 250 described herein. For example, some of the nonmetallic flashing sheets and devices described herein may be used in conjunction with roofing assemblies, around facia, soffets, windows, or along the bottom of siding materials. Furthermore, some of the nonmetallic flashing sheets and devices described herein may be used to wrap or otherwise cover chemically treated wood posts and gutter boards. Because the nonmetallic flashing sheet 200 and device 250 are resistant to corrosion caused the chemicals in the treated lumber, the flashing sheet 200 and device 250 can operate in the aforementioned circumstances for a prolong period of time without substantial corrosion.

A number of embodiments of the invention have been described. Nevertheless, it will be understood that various modifications may be made without departing from the spirit and scope of the invention.

1. A substantially nonmetallic flashing comprising a polymeric material that is substantially resistant to corrosion from chemically treated lumber when in abutment with the chemically treated lumber, the flashing being formed as a length of sheet material releasably retained in a rolled condition, the length of sheet material having a first side and an opposing second side, the first side being a first color and the second side being a second color that is different from the first color, wherein the polymeric material comprises a plasticizer from about 0.1% to about 10% by weight, and wherein the polymeric material has a tensile modulus of from about 250,000 to about 450,000 psi.

2. The flashing of claim 1, wherein the polymeric material comprises PVC from about 60% to about 90% by weight.

3. The flashing of claim 2, wherein the tensile modulus is from about 350,000 to about 400,000 psi.

4. The flashing of claim 3, wherein the plasticizer is an epoxidized vegetable oil.

5. (canceled)

6. The flashing any of claims 2-4, wherein the polymeric material further comprises a light stabilizer from about 3% to about 12% by weight.

7. The flashing of any of claims 2-4, wherein the polymeric material further comprises an acrylic impact modifier or processing aid from about 2.5% to about 10% by weight.

8. The flashing of any of claims 2-4, wherein the polymeric material further comprises a thermal stabilizer from about 0.25% to about 3% by weight.

9. The flashing of any of claims 2-4, wherein the polymeric material further comprises a wax or lubricant additive (or system) from about 0.3% to about 7% by weight.

10. The flashing of claim 6, wherein the light stabilizer is TiO₂.

11. The flashing of claim 8, wherein the thermal stabilizer is an organotin compound.

12. The flashing of claim 9, wherein the one or more waxes or lubricants comprises calcium stearate.

13-16. (canceled)

17. The flashing of claim 1, wherein the first side of the sheet material has a substantially matte finish and the second side has a substantially glossy finish.

18. The flashing of claim 1, wherein the first side of the sheet material has a wood grain finish.

19. The flashing of claim 1, wherein at least a portion of the length of sheet material is releasable from the rolled condition at a jobsite and is cuttable with a handheld cutting blade to a trimmed length.

20. The flashing of claim 3, wherein the polymeric material has coefficient of linear thermal expansion from about 2.5×10⁻⁵ in./in./° F. to about 4.8×10⁻⁵ in./in./° F.

21. A substantially nonmetallic flashing product, comprising:
a roll of sheet material releasably retained in a rolled condition, wherein at least a portion of the length of sheet material is releasable from the rolled condition at a jobsite and is cuttable with a handheld cutting blade to a trimmed length;
the sheet material having a first side and an opposing second side, the first side having substantially matte finish and the second side having a substantially glossy finish;
the flashing product comprising a polymeric material that is substantially resistant to corrosion from chemically treated lumber when in abutment with the chemically treated lumber,
wherein the polymeric material comprises PVC from about 60% to about 90% by weight and comprises a plasticizer in the form of epoxidized vegetable oil from about 0.1% to about 10% by weight.
and wherein the polymeric material has a tensile modulus of from about 350,000 to about 450,000 psi and has coefficient of linear thermal expansion from about 2.5×10⁻⁵ in./in./° F. to about 4.8×10⁻⁵ in./in./° F.

22. The flashing product of claim 21, wherein the first side of the sheet material has a different color appearance than the second side.

23. The flashing product of claim 22, wherein the first side of the sheet material has a wood grain finish.

24. The flashing product of claim 21, wherein the polymeric material comprises one or more light stabilizers from about 0.3% to about 15% by weight, one or more acrylic impact modifiers and/or process aids from about 2.5% to about 10% by weight, one or more thermal stabilizers from about 0.25% to about 3% by weight; and one or more waxes or lubricants from about 0.3% to about 7% by weight.

25. The flashing product of claim 21, wherein the epoxidized vegetable oil is about 7% to about 10%.

26. The flashing product of claim 21, wherein the epoxidized vegetable oil is about 0.1% to about 3%.

27. A method of using a substantially nonmetallic flashing sheet wound into a roll, the flashing sheet comprising a predominantly PVC material and including a first side having a first color appearance that is different than a second color appearance of an opposing second side, the method comprising:
unrolling a length of the flashing sheet from the roll at a job site;
selecting one of the first color appearance of the first side or the second color appearance of the second side to be the outer flashing surface;
cutting at least a portion of the unrolled flashing sheet to a trimmed length at the job site; and
arranging the trimmed length of flashing sheet in abutment with a chemically-treated ledger board mounted to a building at the job site so that the selected outer flashing surface faces outwardly away from the building.

28. The method of claim 27, wherein the at least a portion of the unrolled flashing sheet is cut with a handheld cutter blade.

29. The method of claim 27, wherein the first side of the flashing sheet has a substantially matte finish and the second side has a substantially glossy finish.

30. The method of claim 27, wherein at least one of the first side and the second side of the flashing sheet has a wood grain finish.
